(19) United States
(12) Patent Application Publication
(10) Pub. No.: US 2023/0225765 A1
(43) Pub. Date: Jul. 20, 2023

(54) SPINAL FIXATION ROD HAVING A CORE AND AN OUTER LAYER

(71) Applicant: Medos International Sarl, Le Locle (CH)

(72) Inventors: Jonathan Melchor, Providence, RI (US); Justin Coppes, Franklin, MA (US); Keanan Smith, Mansfield, MA (US); Shawn Emelian, Seekonk, MA (US)

(21) Appl. No.: 17/577,631

(22) Filed: Jan. 18, 2022

(51) Int. Cl. A61B 17/70; A61L 31/08; A61L 31/14; A61L 31/02

(52) U.S. Cl. CPC A61B 17/7011; A61L 31/14; A61L 31/022; A61L 31/088

(57) ABSTRACT

In one embodiment, a spinal fixation rod includes a core and an outer layer. The core extends along a central axis from a first end to a second end and has a length from the first end to the second end sufficient to span two or more adjacent vertebral bodies. The core comprises molybdenum rhenium. The outer layer envelops at least a portion of the core and comprises a biocompatible outer layer material other than molybdenum rhenium.

### BACKGROUND

Spinal fusion involves joining two or more adjacent vertebrae with an anatomical-fixation implant, and more specifically a spinal-fixation implant, to restrict movement of the vertebrae with respect to one another. For a number of known reasons, spinal-fixation implants are used in spine surgery to align and/or fix a desired relationship between adjacent vertebral bodies. Spinal-fixation implants may include, for example, fixation rods and/or fixation plates having sufficient length to span two or more vertebrae and having sufficient rigidity to maintain a fixed relationship between vertebrae under normal physiological loading of the spine. Each fixation rod and/or fixation plate may be attached to the vertebrae via various bone-fixation devices such screws, bolts, nails, hooks or the like, that pass through the rods and/or plates into the vertebrae, or may be attached to the vertebrae via various bone-fixation devices that are attached to the vertebrae before receiving the fixation rods and/or plates, such as bone anchor assemblies having anchor seats with rod-receiving channels.

Some spinal fixation rods are design to be implanted for a sufficient time period to allow the vertebrae to fuse. The implant material should resist corrosion once implanted. Therefore, what is needed is a spinal fixation rod with increased strength and corrosion resistance.

### BRIEF SUMMARY

In one embodiment, a spinal fixation rod includes a core and an outer layer. The core extends along a central axis from a first end to a second end and has a length from the first end to the second end sufficient to span two or more adjacent vertebral bodies. The core comprises molybdenum rhenium. The outer layer envelops at least a portion of the core and comprises a biocompatible outer layer material other than molybdenum rhenium.

### BRIEF DESCRIPTION OF THE DRAWINGS

The foregoing summary, as well as the following detailed description of embodiments of the application, will be better understood when read in conjunction with the appended drawings. For the purposes of illustrating the methods and devices of the present application, there is shown in the drawings representative embodiments. It should be understood, however, that the application is not limited to the precise methods and devices shown. In the drawings:

FIG. 1 is a schematic side elevation view of a bone fixation system constructed in accordance with one embodiment as including a plurality of bone fixation elements attached to underlying bone and connected to each other via a spine fixation rod;

FIG. 2 is an exploded perspective view of one of the bone fixation elements illustrated in FIG. 1;

FIG. 3 is a cross-sectional sectional view of the fixation rod of FIG. 1;

FIG. 4 is a perspective view of the fixation rod of FIG. 1 with a core extending beyond an outer layer;

FIGS. 5A-5D show a plurality of bone fixation elements having a first configuration;

FIGS. 6A-6D show a plurality of bone fixation elements having a second configuration;

FIG. 7 is a side elevational view of a spinal fixation rod with a cap;

FIG. 8 is a cross-sectional side view of the spinal fixation rod and cap of FIG. 7;

FIG. 9 is a side elevational view of a spinal fixation rod with a cap; and

FIG. 10 is a cross-sectional side view of the spinal fixation rod and cap of FIG. 9.

### DETAILED DESCRIPTION

Certain terminology is used in the following description for convenience only and is not limiting. The words “right”, “left”, “lower” and “upper” designate directions in the drawings to which reference is made. The words “inner” and “outer” refer to directions toward and away from, respectively, the geometric center of the bone screw and related parts thereof. The terminology includes the above-listed words, derivatives thereof and words of similar import.

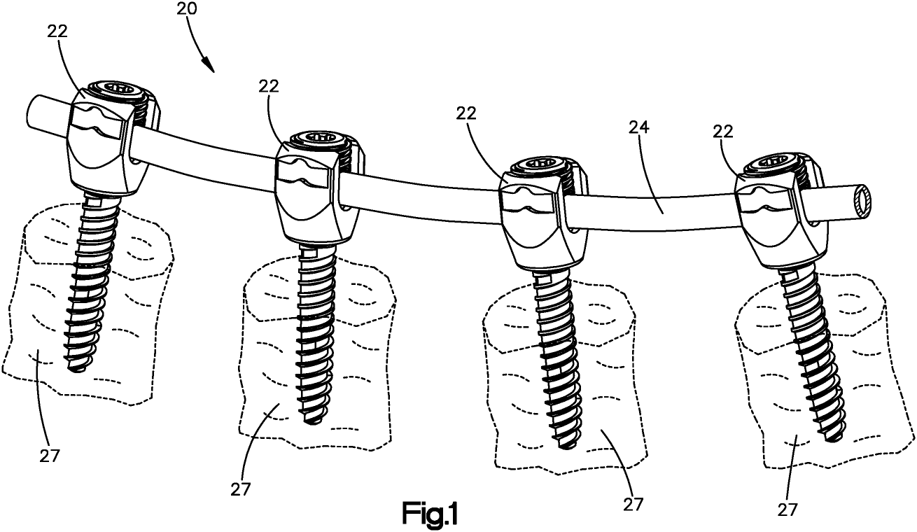

Referring to FIG. 1, a bone fixation assembly 20 includes a plurality of bone fixation elements, such as bone fixation elements 22, connected by a spinal fixation rod 24 that spans between the fixation elements 22. The bone fixation elements 22 can be made from any suitable biocompatible material such as titanium, titanium alloys such as titanium-aluminum-niobium alloy (TAN), implant-grade 316L stainless steel, poly-ether-ether-ketone (PEEK) or any suitable alternative implant-grade material.

The bone fixation elements 22 each include a bone anchor 30 that is implanted into a corresponding vertebra 27 disposed in a spinal region. The spinal region is can include the lumbar region, the thoracic region, or the cervical region as desired. While the spinal fixation rod 24 is illustrated as having a length sufficient to join four bone fixation elements 22, it should be appreciated that the spinal fixation rod 24 can have any length suitable for attachment to any desired number of bone fixation elements configured to attach to any corresponding number of underlying vertebral bodies.

With continuing reference to FIG. 1, the bone fixation elements 22 will be described as and may be generally implanted in the spine, for instance at the pedicle portion of a lumbar, thoracic, or cervical vertebral body. In this regard, when the bone fixation elements 22 are joined by the spinal fixation rod 24, the assembly 20 fixes the relative position of the vertebrae. Accordingly, the bone fixation elements 22 can be referred to as spinal fixation elements or pedicle screw assemblies, the bone fixation assembly 20 can be referred to as a spine fixation assembly.

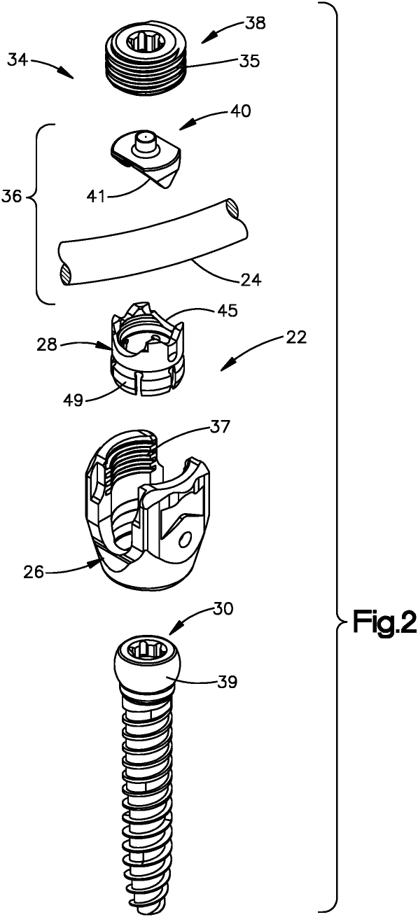

Referring now to FIG. 2, the bone fixation elements 22 generally include a bone anchor seat 26, a collet 28 disposed inside the anchor seat 26, a bone anchor 30 having a head portion 39 attached inside lower fingers 49 of the collet 28, and a locking cap 34 installed in the anchor seat 26 at a location above the collet 28.

The locking cap 34 includes a set screw 38 and a saddle 40 rotatably coupled to the set screw 38. The set screw 38 defines a threaded outer surface 35 that mates with a threaded inner surface 37 of the bone anchor seat 26. The saddle 40 defines a lower surface 41 curved to match that cross-sectional profile of the spinal fixation rod 24. Likewise, the collet 28 defines an upper surface 45 curved to match the cross-sectional profile of the spinal fixation rod. Thus, a rod receiving channel 36 is disposed, and as illustrated defined, between the collet 28 and the locking cap 34. The rod receiving channel 36 is configured to receive the spinal fixation rod 24 therein.

The locking cap 34 can be actuated, such as rotated or screwed, between an unlocked position and a locked position. When the locking cap 34 is in the unlocked position, the spinal fixation rod 24 can slide with respect to the bone fixation elements 22, the bone anchor 30 is free to pivot with respect to the anchor seat 26 as desired, and the bone anchor 30 can further freely rotate relative to the anchor seat 26. When the locking cap 34 is in the locked position, such that the surfaces 41 and 45 bear tightly against the rod 24, the rod 24 is unable to move inside the channel 36, and the collet 28 becomes tightened against the bone anchor such that the bone anchor is unable to pivot or rotate with respect to the collet 28 or the anchor seat 26.

While the fixation assembly 20 has been illustrated in accordance with one embodiment, it should be appreciated that the spinal fixation rod 24 could alternatively extend and connect between fixation elements of any alternatively constructed fixation assembly 20 that is configured to attach or span between to two or more (i.e., a plurality of) underlying vertebral bodies. For instance, while the bone fixation element 22 is illustrated in accordance with one embodiment, the bone fixation element could be described in accordance any alternative embodiment so that it is capable of attaching to the bone spinal fixation rod 24. In this regard, while the bone anchor 30 is illustrated as a bone screw, or pedicle screw, the bone anchor can alternative be provided as a nail, pin, rivet, hook, or any alternatively constructed structure configured to be affixed to the underlying vertebrae.

The fixation elements 22 can exert forces on the spinal fixation rod 24. The spinal fixation rod 24 can have sufficient tensile, compressive, and shear strength to withstand these forces. The spinal fixation rod 24 can have a tensile yield strength of about 400 megapascal (MPa) to about 1,400 MPa, about 400 MPa to about 600 MPa, about 600 MPa to about 800 MPa, about 800 MPa to about 1,000 MPa, about 1,000 to about 1,200 MPa, about 1,200 MPa to about 1,400 MPa, greater than about 400 MPa, greater than about 600 MPa, greater than about 800 MPa, greater than about 1,000 MPa, or greater than about 1,200 MPa. The spinal fixation rod 24 can also be durable such that the spinal fixation rod 24 can have a minimal implant life of at least about 12 months, about 18 months, about 24 months, about 30 months, about 36 months, about 12 months to about 18 months, about 18 months to about 24 months, about 24 months to about 30 months, or about 30 months to about 36 months.

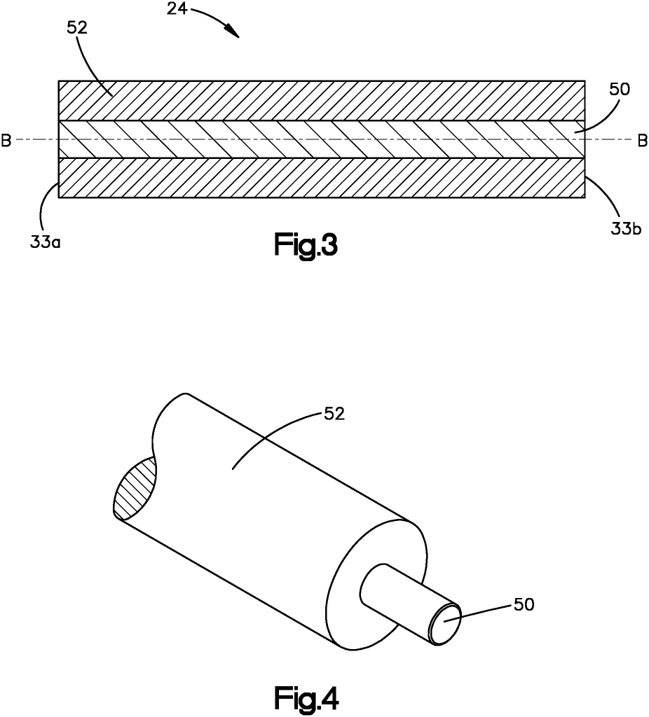

Referring now to FIG. 3, the spinal fixation rod 24 can extend along a central axis B-B from a first end 33a to a second end 33b. The spinal fixation rod 24 may have a length from the first end 33a to the second end 33b of about 30 mm to about 600 mm. One or both of the first end 33a and second end 33b can include a beveled edge, or rounded edge, or bulleted nose. One or both of the first end 33a and second end 33b can be sized and shaped to be engaged by a tool such that the spinal fixation rod 24 can be rotated about the central axis. For example, one of the first end 33a and second end 33b can be defined by a hexagon shape, an oval shape, or two opposing flat surfaces configured to be engaged by a tool to rotate the spinal fixation rod about the central axis.

The spinal fixation rod 24 can include a core 50 that extends from the first end 33a to the second end 33b. The core 50 may form the portion of the spinal fixation rod 24 that is configured to be engaged by the tool.

The core 50 may be manufactured from molybdenum rhenium. Thus, the core 50 can include a quantity of molybdenum rhenium, or in some examples can be a molybdenum rhenium core. The core 50 may consist essentially of molybdenum rhenium. A core that consists essentially of molybdenum rhenium may include trace amounts of other materials without significantly impacting performance (e.g., strength) of the core 50. The core 50 may consist of only molybdenum rhenium. The core 50 may include at least 50%, at least 60%, at least 70%, at least 80%, at least 90%, at least 95%, at least 99%, or at least 99.9% molybdenum rhenium by weight, by volume, or by molecular quantity. The core 50 may include molybdenum rhenium and trace amounts of another material. The core 50 may include molybdenum rhenium and trace amounts of carbon, oxygen, hydrogen, copper, manganese, silicon, titanium, or iodine. The core 50 may include about 20%, about 30%, about 40%, about 50%, about 60%, about 70%, or about 80% molybdenum. The core 50 may include about 20%, about 30%, about 40%, about 50%, about 60%, about 70%, or about 80% rhenium.

The core 50 may have a circular cross-sectional length when taken along a plane perpendicular to the central axis. Alternatively, the core 50 may have an oval, octagon, hexagon, triangle, square, or any suitable shape cross section. The core 50 may have a maximum cross-sectional dimension of about 2.5 millimeters to about 3.5 millimeters, about 3.5 millimeters to about 4.5 millimeters, about 4.5 millimeters to about 5.5 millimeters, or about 5.5 millimeters to about 6.5 millimeters taken along a plane perpendicular to the central axis B-B. The core 50 may have a maximum cross-sectional area of about 5 square millimeters to about 10 square millimeters, about 10 square millimeters to about 15 square millimeters, about 15 square millimeters to about 20 square millimeters, about 20 square millimeters to about 25 square millimeters, about 25 square millimeters to about 30 square millimeters, or about 30 square millimeters to about 35 square millimeters taken along a plane perpendicular to the central axis B-B.

A core 50 manufactured from molybdenum rhenium can be strong enough to stabilize the vertebrae for a sufficient length of time that allows the vertebrae to fuse, for instance to an interbody spacer. However, the present inventors have discovered molybdenum rhenium may corrode over time when implanted into the patient and exposed to blood or other bodily fluids. Therefore, referring to FIGS. 3 and 4, the spinal fixation rod 24 can include an outer layer 52 on the core 50 that provides a barrier to inhibit or prevent exposure of the core 50 to blood or other bodily fluids. Thus, the outer layer 52 can reduce corrosion of the core 50 to levels that indicate biocompatibility, or can prevent corrosion of the core 50 altogether.

The outer layer 52 can envelope at least a portion of the core 50. The outer layer 52 can be a coating. In other embodiments, the outer layer 52 can be a sleeve or layer that is coupled to the core 50. The outer layer 52 can prevent oxidation of the portion of the core 50 enveloped by the outer layer 52. For example, the outer layer 52 may form a substantially impervious barrier to prevent contact between the core 50 and at least one of gas and fluid inside the body. In some embodiments, a substantially impervious barrier can prevent fluid or gas from passing through the barrier. In other embodiments, a substantially impervious barrier can prevent fluid from passing through the barrier.

The outer layer 52 can extend in a circumferential direction about an outer surface of the core 50. In one example, the outer layer 52 can extend continuously about the outer surface of the core 50 in the circumferential direction. The outer layer 52 can have an uninterrupted outer surface that extends the outer surface of the core 50 from a first end of the outer layer 52 to a second end of the outer layer 52 opposite the first end. Alternatively, the outer layer 52 may include one or more openings such that a portion of the core 50 remains externally exposed. In some embodiments, the outer layer 52 does not envelop the ends of the core 50. In other embodiments, the outer layer 52 envelops the ends of the core 50. The outer layer 52 can cover at least about 40% to about 50%, about 50% to about 60%, about 60% to about 70%, about 70% to about 80%, about 80% to about 90%, or about 90% to about 100% percent of an outer surface area of the core.

The outer layer 52 may have a shorter length than the core 50 such that a portion of the core 50 extends beyond at least one end of the outer layer 52 as shown in FIG. 4. In other embodiments the ends of the outer layer 52 are aligned with the ends of the core 50. For example, the outer layer 52 and the core 50 can be equal length. The outer layer 52 may have a thickness of about 1 micron to about 100 microns as measured in a direction perpendicular to the central axis. The outer layer 52 can have an outer diameter of about 3.5 mm to about 6.5 mm.

The outer layer 52 can be any suitable biocompatible outer layer material. The outer layer material can be a material other than molybdenum rhenium. The outer layer 52 can be substantially devoid of molybdenum rhenium. The outer layer 52 can include less than 25%, less than 20%, less than 15%, less than 10%, less than 5%, less than 1%, or less than 0.1% molybdenum rhenium by weight, by volume, or by molecular quantity. The outer layer 52 can be made from a biocompatible material such as titanium, commercially pure titanium (cpTi), titanium alloys such as titanium-aluminum-niobium alloy (TAN), titanium-aluminum-vanadium (TAV), titanium-nitrogen (TiN), titanium-implant-grade 316 L stainless steel, poly-ether-ether-ketone (PEEK), chromium-nitrogen (CrN), diamond like carbon (DLC), or any suitable alternative implant-grade material. The outer layer 52 can consist essentially of at least one of titanium, titanium alloy, and stainless steel. An outer layer that consists essentially of at least one of titanium, titanium alloy, and stainless steel may include trace amounts of other materials without significantly impacting performance (e.g., resistance to corrosion) of the outer layer.

As explained below, the outer layer 52 can be a physical vapor deposition (PVD) or an ion beam enhanced deposition (IBED) on the outer surface of the core 50. Alternatively, the outer layer 52 can be a chemical vapor deposition (CVD), chemical vapor aluminizing (CVA), or plasma assisted chemical vapor deposition (PACVD).

The spinal fixation rod 24 can be more corrosion resistant than the isolated core 50. The spinal fixation rod 24 can have a corrosion rate of about 1.0 µm per year under potentiodynamic testing in accordance with ASTM F2129-19A in de-aerated phosphate buffered saline at about 37° C. One method of potentiodynamic testing can include cyclic (forward and reverse) potentiodynamic polarization. For example, the method can include immersing the spinal fixation rod 24 in a solution (e.g., a phosphate buffered solution having a pH of about 6 to about 8). The solution can be purged of oxygen by diffusing nitrogen gas into the solution (e.g., at a flow rate of about 150 cm³/min) before and during the test procedure. An electrical wire can be connected to the spinal fixation rod 24 and to a reference electrode. The spinal fixation rod 24 and the reference electrode can both be submerged in the solution as well as a salt bridge probe. A voltage can then be applied to the electrical wires. The applied voltage can be changed during the test procedure. The scan rate, or rate of change in the voltage, can be 0.167 mV/s or 1 mV/s. After the test is complete, corrosion can be determined based on changes in weight, volume, or physical appearance of the spinal fixation rod 24.

The spinal fixation rod 24 can be flexible. The spinal fixation rod can have a modulus of elasticity of about 200 to about 400 gigapascals (Gpa). The spinal fixation rod 24 can be configured to resist fatigue to prolong the life of the spinal fixation rod. The spinal fixation rod 24 can include a greater resistance to fatigue than a cobalt chromium fixation rod having the same dimensions as the spinal fixation rod 24.

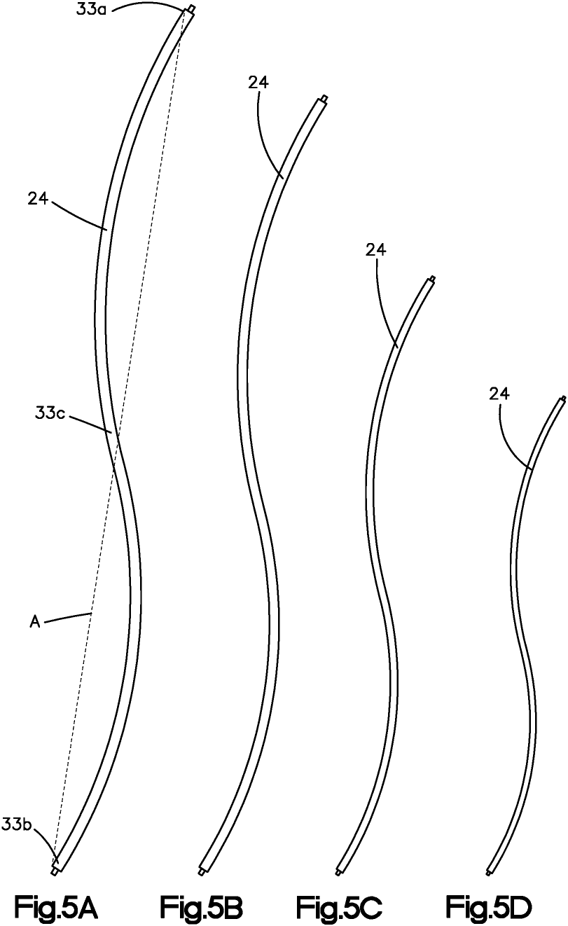

Referring to FIGS. 5, the spinal fixation rod 24 can extend along a first path from the first end 33a to the second end 33b. The first path can include a precontoured bend. The precontoured bend may include a radius of curvature of about 150 mm to about 400 mm. The spinal fixation rod 24 can include a curved profile 31 defined at least in part by the first and second ends 33a and 33b, and a middle portion 33c disposed between the first end 33a and second end 33b and offset with respect to an imaginary straight-line A that joins first end 33a and second end 33b. In the illustrated embodiment, the middle portion 33c is disposed posterior with respect to the first end 33a and second end 33b when the bone fixation elements 22 are implanted into the spine, such that the rod 24 is convex with respect to the spinal column 23, though it should be appreciated that the spinal fixation rod 24 could be curved when implanted such that the middle portion 33c is disposed anteriorly with respect to the first end 33a and second end 33b, such that the spinal fixation rod 24 is convex with respect to the spinal column. Accordingly, in the illustrated embodiment, the spinal fixation rod 24 is curved such that the first end 33a and second end 33b and the middle portion 33c, lie in a desired plane, such as the sagittal plane S, when the bone fixation assembly 20 is secured to the underlying vertebrae 27. The spinal fixation rod 24 can include at least one of a lordotic curve and a kyphotic curve. In this regard, the spinal fixation rod 24 is configured to impart at least one of a lordotic profile and a kyphotic profile onto the underlying vertebrae 27.

Referring to FIGS. 5, the spinal fixation rod 24 can be a generally straight member. The spinal fixation rod 24 can be configured to be bent from a first configuration to a second configuration. The spinal fixation rod 24 may be bent from the first configuration to the second configuration intraoperatively. The spinal fixation rod 24 can extend from the first end 33a to the second end 33b along a first path when the spinal fixation rod 24 is in the first configuration. The spinal fixation rod 24 can extend from the first end 33a to the second end 33b along a second path when the spinal fixation rod 24 is in the first configuration. The second path can be different from the first path. Methods of bending spinal fixation rods are disclosed in U.S. Pat. 10,390,884 which is attached hereto as Appendix A.

An anatomical fixation kit may include the spinal fixation rod 24. The kit can include two or more vertebral fixation elements 22. The vertebral fixation elements 22 may be manufactured from a material other than molybdenum rhenium.

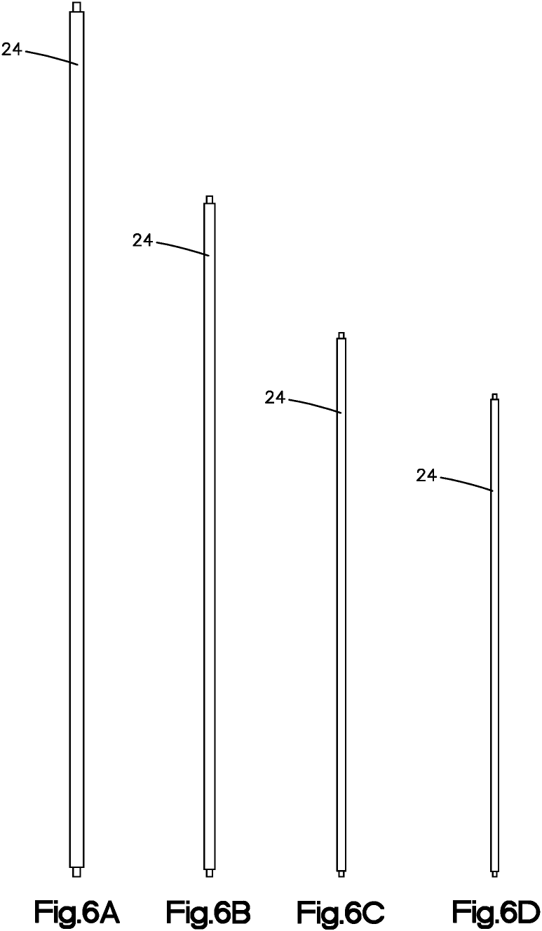

Referring to FIGS. 5 and 6, the anatomical fixation kit may include a plurality of spinal fixation rods 24. One or more of the spinal fixation rods 24 may have a physical characteristic that is different from another of the spinal fixation rods 24. For example, one spinal fixation rod 24 may have a different length or thickness than another of the spinal fixation rods 24. One of the spinal fixation rods 24 can have a precontoured bend that is different than the precountoured bend of at least one of the other of the plurality of spinal fixation rods 24.

The kit can include one or more spinal fixation rods 24 having the precontoured bend. The kit can include one or more generally straight spinal fixation rods 24. The kit can include a combination of one or more generally straight spinal fixation rods 24 and one or more spinal fixation rods 24 having a precontoured bend.

The spinal fixation rod 24 may be cut to a desired length with a saw, laser, or other cutting instrument. A portion of the core 50 may be exposed at one or both of the first end 33a and second end 33b when the rod is cut. It may be desirable to seal the first end 33a and second end 33b after the spinal fixation rod 24 is cut.

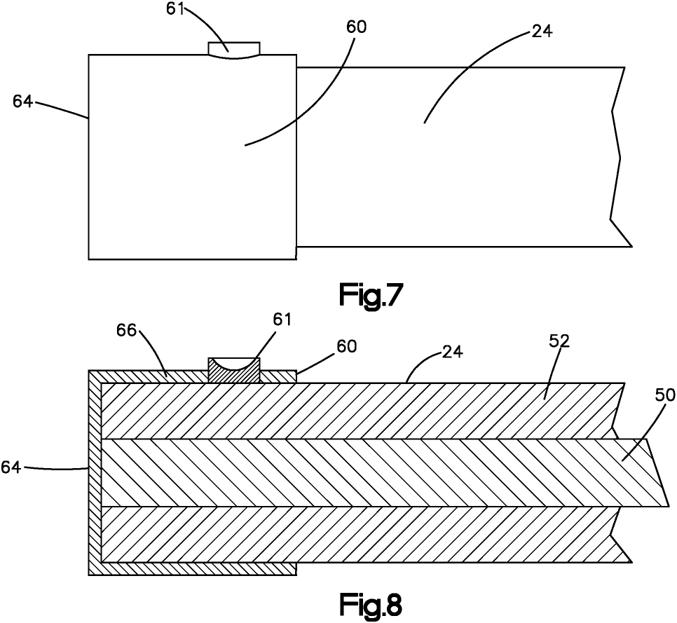

Referring to FIGS. 7-8, a cap 60 may be coupled to the spinal fixation rod 24. The cap 60 can provide a barrier to inhibit or prevent exposure of the core 50 to blood or other bodily fluids. The cap 60 can be configured to envelop an exposed end of the core 50.

The cap 60 may include an endwall 64. The endwall 64 can cover an end of the spinal fixation rod 24. In some embodiments, the endwall 64 contacts an end surface of the spinal fixation rod 24. In other embodiments, the endwall 64 is spaced from an end of the spinal fixation rod.

A sidewall 66 can be coupled to the endwall 64. The sidewall 66 can extend in a circumferential direction about an outer surface of the outer layer 52. In one example, the sidewall 66 can extend continuously about the outer surface of the outer layer 52 in the circumferential direction.

The cap 60 may define a recess to receive a portion of the spinal fixation rod 24. The recess may be defined by an inner surface of the sidewall 66. In some embodiments, the recess is slightly larger than the outer dimension of the spinal fixation rod 24 such that the cap 60 can be positioned over the end of the rod 24. For example, cap 60 may be manually positioned on an end of the spinal fixation rod 24. In other embodiments, the recess is slightly smaller than the outer dimension of the spinal fixation rod 24 such that the cap 60 can be press fit onto the end of the rod 24.

A fastener can secure the cap 60 to the spinal fixation rod 24. Referring to FIGS. 7-8, the fastener can be a threaded fastener such as a set screw 61. The outer layer 52 or core 50 can include a recess to recess to receive a portion of the set screw 61. The recess can include a thread to threadedly engage the set screw 61. Alternatively, the fastener could be a worm gear style fastener, a magnet, or a rivet. In other embodiments, cap 60 is coupled to spinal fixation rod 24 without a fastener. For example, cap 60 could be secured to spinal fixation rod 24 via adhesive, welding, threaded connection, or press fit.

An o-ring (not shown) may be positioned between the cap 60 and the spinal fixation rod 24. The o-ring can be positioned between the spinal fixation rod 24 and one or more of the sidewall 66 and the endwall 64. The o-ring may provide a seal between the cap 60 and the spinal fixation rod 24 even when the one or both of the endwall 64 and sidewall 66 are spaced from the outer surface of the spinal fixation rod 24. The cap 60 can include more than one o-ring.

In some embodiments, the ends of the core 50 and outer layer 52 are generally flush after cutting the spinal fixation rod 24 and the cap 60 is positioned over both the outer layer 52 and the core 50 as shown in FIG. 8. In other embodiments, a portion of the core 50 extends beyond an end of the outer layer 52 such that the end of the core 50 is exposed as shown in FIG. 4 and the cap 60 can be coupled to the exposed portion of the core 50. An inner surface of the cap 60 may be coupled to an outer surface of the outer layer 52. An outer surface of the cap 60 can be generally flush with the outer surface of the outer layer 52. For example, the sidewall 66 and outer layer 52 can have a similar thickness such that the outer surface of the cap 60 is generally flush with the outer surface of the outer layer 52 when the cap 60 is coupled to an exposed portion of the core 50.

The cap 60 can be made of a biocompatible material such as titanium, titanium alloys such as titanium-aluminum-niobium alloy (TAN), titanium-aluminum-vanadium (TAV), titanium-nitrogen (TiN), titanium-implant-grade 316L stainless steel, poly-ether-ether-ketone (PEEK), chromium-nitrogen (CrN), diamond like carbon (DLC), or any suitable alternative implant-grade material. The cap 60 can consist essentially of at least one of titanium, titanium alloy, and stainless steel. A cap that consists essentially of at least one of titanium, titanium alloy, and stainless steel may include trace amounts of other materials without significantly impacting performance (e.g., resistance to corrosion) of the cap.

The cap 60 can include an inner core of molybdenum rhenium and an outer layer of biocompatible material such as commercially pure titanium (cpTi), titanium-aluminum-niobium alloy (TAN), titanium-aluminum-vanadium (TAV), titanium-nitrogen (TiN), titanium-implant-grade 316L stainless steel, poly-ether-ether-ketone (PEEK), chromium-nitrogen (CrN), diamond like carbon (DLC), or any suitable alternative implant-grade material. A cap that includes a non-metallic biocompatible material can be resistant to bimetallic corrosion.

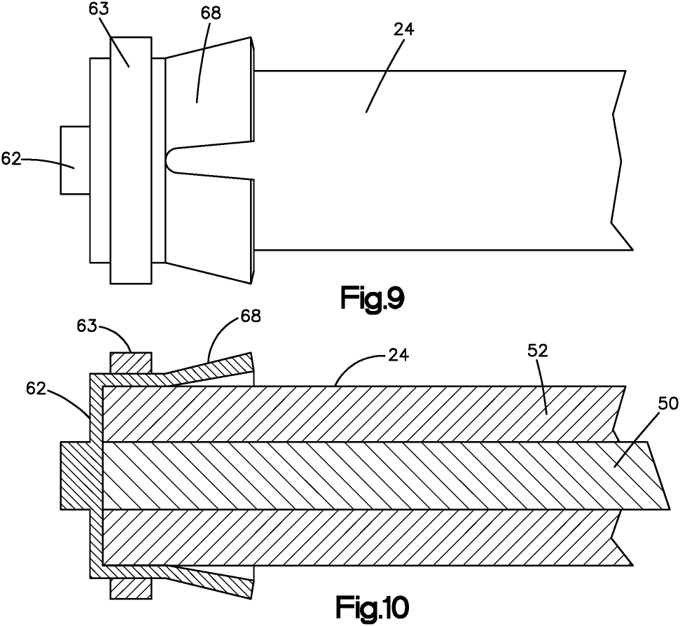

Referring to FIGS. 9-10, another embodiment of a cap 62 is shown. Cap 62 can be similar to cap 60, but cap 62 can include a collet to engage the spinal fixation rod 24. Cap 62 can include a sidewall 68 that engages the spinal fixation rod 24 as the collet is tightened. A restraint 63 can engage the cap 62 to tighten the sidewall 68 into engagement with the spinal fixation rod 24, thereby fixing the cap 62 to the rod 24. Sidewall 68 can include a flared profile such that movement of the restraint 63 relative to the sidewall 68 can constrict the sidewall 68 about the spinal fixation rod 24. Restraint 63 can be an external member that engages an outer surface of the sidewall 68. For example, restraint 63 can be a hex nut that threadedly engages the cap 62 to constrict the sidewall 68 about the spinal fixation rod 24. Restraint 63 could be threadless and moved by an external instrument to force restraint 63 over sidewall 68. In other embodiments, restraint 63 can be an internal feature. Cap 62 can include an engagement feature 65 engageable by a tool. For example, engagement feature 65 can be a hex nut that can be engaged by a wrench to move the cap 62 and restraint 63 relative to each other. Alternatively, engagement feature 65 can be a recess sized and dimensions to receive a screw driver or hex key.

A method of manufacturing a spinal fixation rod 24 can include coating at least a portion of the core 50 with an outer layer material so as to define the outer layer 52 that envelops at least a portion of the core 50. The coating step can include at least one of physical vapor deposition (PVD), ion beam enhanced deposition (IBED), additive manufacturing, and three-dimensional printing. The method may include anodizing, passivation, or etching the outer layer 52.

A PVD process can be a vacuum coating process wherein the outer layer 52 is deposited atom by atom on the core 50 by condensing the outer layer 52 from a vapor phase to a solid phase. The PVD process can include at least one of sputtering and evaporation. The temperature for the PCD process can be about 150° C. to about 180° C.

An IBED process can include simultaneously bombarding the core 50 with a beam of energetic atomic particles and a growing film. The growing film can be generated by vacuum evaporation of the outer layer material. The beam of energetic atomic particles can include charged atoms of at least one of neon, argon, krypton, nitrogen, or oxygen.

The method may include precleaning the core 50 before the coating step. The precleaning step can include at least one of pickling, electropolishing, and blasting. Precleaning can help remove oxides on an outer surface on the core 50.

The method can include cutting the spinal fixation rod 24 to a desired length. The method can include coupling a cap to the spinal fixation rod 24. The coupling step may include adjusting a fastener to secure the cap to the spinal fixation rod 24. The fastener can be a set screw 61 or a restraint 63. Adjusting the fastener can include at least one of rotating, sliding, pushing, or pulling the fastener.

A method of manipulating the spinal fixation rod 24 can include bending the spinal fixation rod 24 from a first configuration whereby the spinal fixation rod extends from the first end to the second end along a first path, to a second configuration whereby the spinal fixation rod extends from the first end to the second end along a second path different from the first path. The method can include placing the spinal fixation rod 24 adjacent an anatomical body prior to the bending step. The bending step can include bending the spinal fixation rod with a computer controlled bending machine. Alternatively, or in addition, the bending step can include bending the spinal fixation rod with a mandril.

It will be appreciated by those skilled in the art that changes could be made to the embodiments described above without departing from the broad inventive concept thereof. Furthermore, it should be appreciated that the structure, features, and methods as described above with respect to any of the embodiments described herein can be incorporated into any of the other embodiments described herein unless otherwise indicated. It is understood, therefore, that this invention is not limited to the particular embodiments disclosed, but it is intended to cover modifications within the spirit and scope of the present disclosure. Further, it should be appreciated, that the term substantially indicates that certain directional components are not absolutely perpendicular to each other and that substantially perpendicular means that the direction has a primary directional component that is perpendicular to another direction.

1. A spinal fixation rod comprising:
a core that extends along a central axis from a first end to a second end and has a length from the first end to the second end sufficient to span two or more adjacent vertebral bodies, wherein the core comprises molybdenum rhenium;
an outer layer that envelops at least a portion of the core, the outer layer comprising a biocompatible outer layer material other than molybdenum rhenium; and
a cap defining a recess with at least a portion of the core positioned within the recess so as to envelop the core and prevent exposure of the core to bodily fluids.

2. The spinal fixation rod of claim 1, wherein the core comprises at least 99% molybdenum rhenium.

3. The spinal fixation rod of claim 1, wherein the outer layer material is substantially devoid of molybdenum rhenium.

4. The spinal fixation rod of claim 1, wherein the outer layer material comprises at least one of titanium, a titanium alloy, and stainless steel.

5. The spinal fixation rod of claim 1, wherein the outer layer comprises a physical vapor deposition or an ion beam enhanced deposition.

6. The spinal fixation rod of claim 1, wherein the first end includes at least one of a hexagon shape, an oval shape, and two opposing flat surfaces configured to be engaged by a tool to rotate the spinal fixation rod about the central axis.

7. The spinal fixation rod of claim 1, wherein the spinal fixation rod extends along a first path from the first end to the second end, the first path including a precontoured bend.

8. The spinal fixation rod of claim 7, wherein the precontoured bend includes at least one of a lordotic curve and a kyphotic curve.

9. The spinal fixation rod of claim 1, wherein the spinal fixation rod is configured to be bent from a first configuration whereby the spinal fixation rod extends from the first end to the second end along a first path, to a second configuration whereby the spinal fixation rod extends from the first end to the second end along a second path different from the first path.

10. The spinal fixation rod of claim 1, wherein the spinal fixation rod includes a modulus of elasticity of 200 to 400 gigapascals.

11. The spinal fixation rod of claim 1, wherein the outer layer prevents oxidation of the portion of the core enveloped by the outer layer.

12. The spinal fixation rod of claim 1, wherein the outer layer covers at least 60 percent of an outer surface area of the core.

13. The spinal fixation rod of claim 1, wherein the spinal fixation rod comprises a tensile strength of at least 1,000 megapascals.

14. (canceled)

15. The spinal fixation rod of claim 1, wherein the spinal fixation rod comprises a corrosion rate of about 1.0 µm per year under potentiodynamic testing in accordance with ASTM F2129-19A in de-aerated phosphate buffered saline at about 37° C.

16. The spinal fixation rod of claim 1, wherein the outer layer has a thickness of 1 micron to 100 microns as measured in a direction perpendicular to the central axis.

17. An anatomical fixation kit comprising:
the spinal fixation rod of claim 1; and
two or more adjacent vertebral fixation elements comprising a vertebral fixation element material different other than molybdenum rhenium.

18. The anatomical fixation kit of claim 17, wherein the vertebral fixation element material comprises at least one of titanium, cobalt chrome, and stainless steel.

19. The anatomical fixation kit of claim 17, wherein the vertebral fixation elements each include a channel configured to receive the spinal fixation rod.

20. The anatomical fixation kit of claim 19, wherein the spinal fixation rod is one of a plurality of spinal fixation rods and each of the plurality of spinal fixation rods includes a precontoured bend that is different than a precountoured bend of at least one of the other of the plurality of spinal fixation rods.
